(19) United States
(12) Patent Application Publication
(10) Pub. No.: US 2026/0097523 A1
(43) Pub. Date: Apr. 9, 2026

(54) END-EFECTOR FOR ROBOTIC ARMS

(71) Applicant: MULTI-SCALE MEDICAL ROBOTICS CENTER LIMITED, Hong Kong (CN)

(72) Inventors: Ka Chun LAU, Hong Kong (CN); Lap Wing CHEUNG, Hong Kong (CN); Yeung YAM, Hong Kong (CN); Wai Yan Philip CHIU, Hong Kong (CN)

(21) Appl. No.: 19/120,769

(22) PCT Filed: Oct. 12, 2023
(86) PCT No.: PCT/IB2023/060261

§ 371 Date: Apr. 12, 2025

Related U.S. Application Data
(60) Provisional application No. 63/415,640, filed on Oct. 12, 2022.

(51) Int. Cl. B25J 15/00; B25J 9/10

(52) U.S. Cl. CPC B25J 15/0033; B25J 9/104

(57) ABSTRACT

This invention provides an end-effector for robotic arms. In one embodiment, said adaptor comprises: a) a first housing having a center line; b) at least one rotational member with rotational axis perpendicular to said center line; c) a first pair of actuation wires; wherein said first pair of actuation wires are connected to said at least one rotational member to actuate rotation with a pull-pull mechanism whereby tension and displacement in each wire of said first pair of actuation wires are same but direction is different.

### FIELD OF THE INVENTION

The present invention generally relates to end-effector for robotic arms, particularly non-back driven cable driven grippers.

### BACKGROUND OF THE INVENTION

Gastric and colorectal cancers are the common types of cancer in different area of the world. They are also the top leading causes of cancer death. Gastrointestinal (GI) cancers grow from mucosal layer. Survival rates for patients suffering from these cancers may be improved if premalignant and early cancers are removed before they spread to lymph nodes.

Endoscopic Submucosal Dissection (ESD) and Endoscopic Mucosal Resection (EMR) were developed for removal of pre-malignant and early caners in the GI tract. These procedures are performed with the use of a flexible endoscope and have advantages of being minimally invasive and organ sparing.

ESD or EMR closure requires precise suturing to avoid perforation and bleeding. In open surgery, a classical approach to suturing is to manipulate the suturing needle with a long straight gripping instrument. It has a lobster-liked gripper and several rotary joints driven by actuation wires such as Shape Memory Alloy (SMA). When penetrating the needle into tissue, the gripper rotates its wrist so that the needle follows a circular trajectory. Mechanical movement can be conveyed easily with a shaft from the controller. This intuitive design reduces learning curve of surgeons.

However, developing reliable endoscopic suturing devices has been a long challenge for engineers because of its critical dimension and strength requirement. In general, endoscopic suturing robot has the following difficulties. First, the robotic arm must be small and flexible enough to reach the target position without hurting the patient. Second, the complexity and number of driving parts of the suturing mechanism is limited since current flexible endoscopes usually have only one to two instrument channels. Third, the robot should manipulate the suturing needle firmly that maintain its position and orientation. Forth, rotation along endoscope's axis is technically demanding because transferring rotational movement outside patient's body to the gripping device is difficult.

### SUMMARY OF THE INVENTION

This invention provides an end-effector for robotic arms. In one embodiment, said adaptor comprises: a) a first housing having a center line; b) at least one rotational member with rotational axis perpendicular to said center line; c) a first pair of actuation wires; wherein said first pair of actuation wires are connected to said at least one rotational member to actuate rotation with a pull-pull mechanism whereby tension and displacement in each wire of said first pair of actuation wires are same but direction is different.

### BRIEF DESCRIPTION OF THE FIGURES

FIG. 1 shows the schematic diagram illustrating the inner structure of an embodiment of the end-effector of this invention.

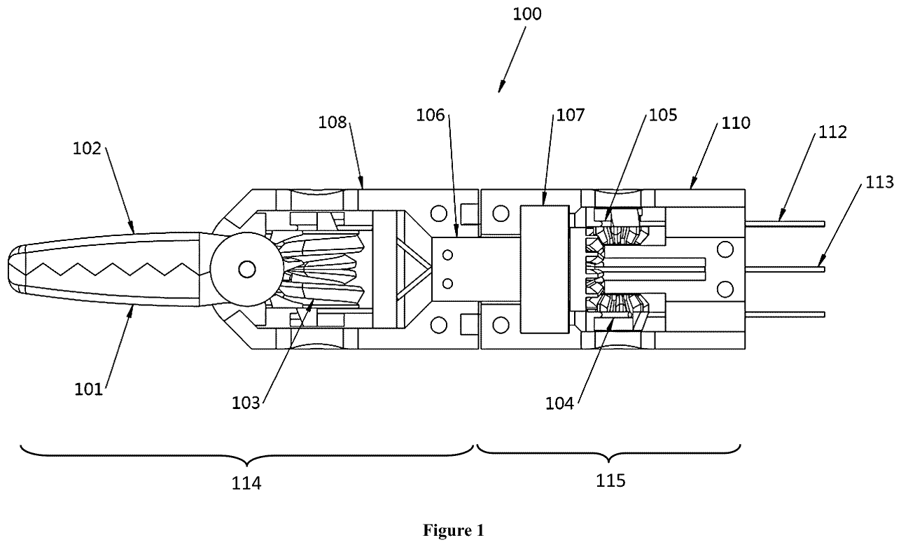

FIG. 2 shows the schematic diagram of the exploded view of the embodiment in FIG. 1.

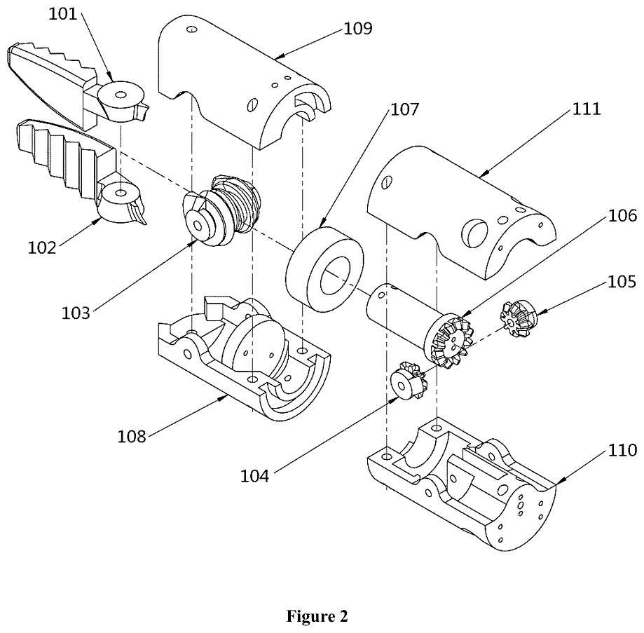

FIG. 3 shows the schematic diagram of the interlocking mechanism between the globoid worm gear and the gripper jaws in the embodiment of FIG. 1.

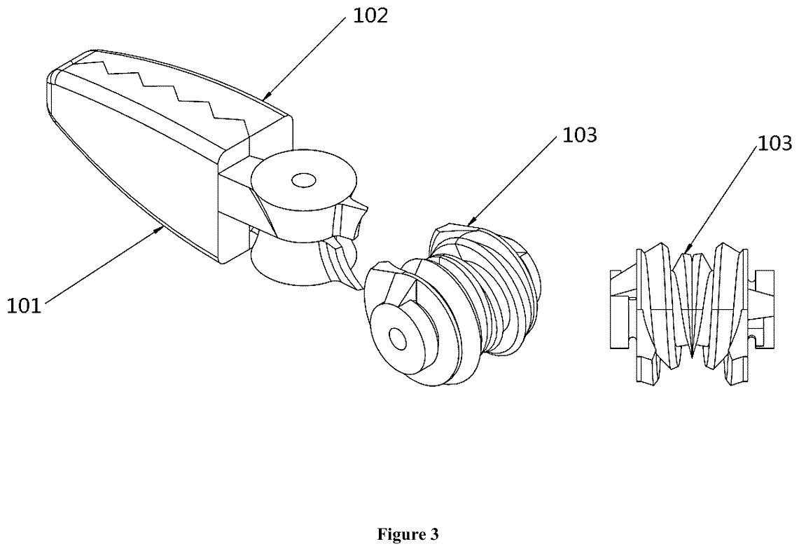

FIG. 4 shows the schematic diagram of the driving bevel gear in the embodiment of FIG. 1.

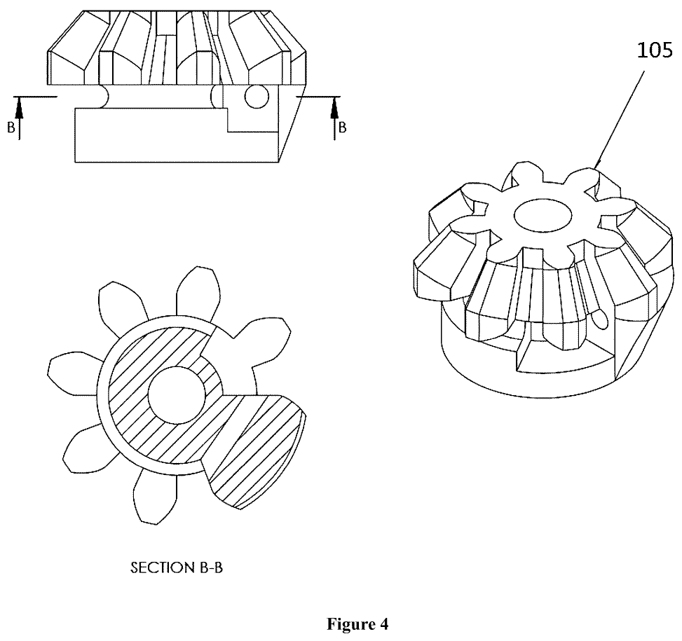

### DETAILED DESCRIPTION OF THE INVENTION

Described herein is the structure and method of a non-back driven cable driven gripper to be used as an end-effector in the robotic system. The fundamental application of this gripper is to provide a clinical solution to endoscopic suturing. The non-back driven gripper provides an extraordinarily high gripping force to the suturing needle; and the rotary joint provides a rotational motion to penetrate the tissue, at which these two structures are embedded into a tiny space. These two structures are motorized by a pair of pulling wire which favor the gripper can be installed in any cable driven robotic arm. Scalable is also the advantage of this gripper to enable it can be applied to many applications, not limited to endoscopic surgery.

In one embodiment, the present invention provides a non-back driven cable driven gripper, said non-back driven cable driven gripper is a task specific device for the procedure of endoscopic suturing. This device has two degree-of-freedoms (DOF), a gripper and a rotary joint along the body axis, which can be attached to flexible robotic arm. An optimal design of this distal end device requires the use of coated steel wire actuators to improve actuation efficiency and control accuracy. In one embodiment, this invention generally relates to endoscopic robots, more particularly, to suturing application in endoscopic surgery with flexible robotic arm.

This invention provides a structure for gripper to be used in robotic system. In one embodiment, said structure comprises: a first member of a rotation section, with two bevel gears with mirrored shape mounted inside a housing with its rotational axis perpendicularly to the center line of the housing; at which these two bevel gears are connected with another center bevel gear which the rotation axis of this gear is collided with the center line of the housing simultaneously; a second member of a gripper section, with the housing this section is connected to the end of the bevel gear from the first member for syncing the rotational motion; at which a globoid worm gear is mounted perpendicularly to the center line of the housing, such that the gripper jaws with a small gear tooth connected with the globoid worm gear to form a globoid spiral motion for driving the gripper jaws.

In one embodiment, the two bevel gears are connected to one of a pair of actuation wire, which these wires are configured in a pull-pull mechanism such that they are actuated in different direction but with the same displacement.

In one embodiment, the two bevel gears are rotated in different directions by pulling the corresponding actuation wire.

In one embodiment, the center bevel gear is rotated according to the direction of rotation of the bevel gears actuated by the pair of actuation wires.

In one embodiment, whereby the center bevel gear is actuated in rotary motion by actuating a pair of actuation wire in pull-pull configuration.

In one embodiment, the bevel gear has wiring grove under the gear face for anchoring the end of the actuation wire.

In one embodiment, the housing is designed into two pieces, and they can be connected by using metal pins.

In one embodiment, the center bevel gear are supported and rotates freely in the circular bearing, at which the bearing is located in the second member.

In one embodiment, the gripper section housing is connected with the center bevel gear in member 1 by using metal pins.

In one embodiment, whereby member 2 rotates with the center bevel gear in member 1 simultaneously.

In one embodiment, the gripper section rotates along the center of the housing by actuating the pair of actuation wire in member 1.

In one embodiment, the gripper section has another pair of actuation wire passing the hole in the center of member 1.

In one embodiment, the pair of actuation wire is connected to the opposite side of the globoid worm gear.

In one embodiment, two gripper jaws are connected perpendicularly to the center line of the housing.

In one embodiment, the two gripper jaws have a small gear attached at the edge of the joint and connect to the gear surface of globoid worm gear.

In one embodiment, the gripper jaws rotate in opposite direction from the rotation motion of the globoid gear.

In one embodiment, the gripper jaws is actuated by the pair of actuation wire passing through the hole in center of the center bevel gear in member 1 independently from the rotation motion of member 1.

In one embodiment, the gear teeth of the gripper mating with the globoid worm gear are shifted upward and downward from the center line.

In one embodiment, the globoid worm gear has two sets of tongue and grove in opposite direction.

In one embodiment, four wires from the flexible robotic arm are anchored at the opening of circular housing.

This invention provides an end-effector for robotic arms. In one embodiment, said adaptor comprises: a) a first housing having a center line; b) at least one rotational member with rotational axis perpendicular to said center line; c) a first pair of actuation wires; wherein said first pair of actuation wires are connected to said at least one rotational member to actuate rotation with a pull-pull mechanism whereby tension and displacement in each wire of said first pair of actuation wires are same but direction is different.

In one embodiment, said at least one rotational member comprises two bevel gears (104, 105), each of said two bevel gears is separately connected to one of said first pair of actuation wires (112) to form said pull-pull mechanism, said two bevel gears connects to a central bevel gear (106) with a rotational axis along said center line.

In one embodiment, said at least one rotational member comprises a globoid worm gear (103) having two ends, wherein each of said two ends is separately connected to one of said first pair of actuation wires (113) to form said pull-pull mechanism.

In one embodiment, said globoid worm gear (103) comprises an hourglass surface having two sets of tongues and groves interlocking with mating components on gripper jaws (101, 102).

In one embodiment, said central bevel gear (106) is connected to an end part to actuate rotation of said end part.

In one embodiment, said end part comprises a second housing and a second pair of actuation wires, wherein said second housing contains a globoid worm gear (103) having two ends, wherein each of said two ends is separately connected to one of said second pair of actuation wires (113) to form a pull-pull mechanism.

In one embodiment, said globoid worm gear (103) comprises an hourglass surface interlocking with mating components on two gripper jaws (101, 102) to drive angle of said two gripper jaws.

In one embodiment, said first housing comprises a tongue for insertion into a grove on said second housing to act as a stopper.

In one embodiment, said two bevel gears comprises a gear face and a wiring grove, wherein said wiring grove is under said gear surface.

In one embodiment, said gripper jaws are connected to said end-effector with one or more metal pins.

In one embodiment, said mating components comprises a small gear.

In one embodiment, said hourglass surface comprises two sets of tongues and groves, each of said two sets of tongues and groves being in opposite direction of the other.

In one embodiment, said two gripper jaws comprises gear teeth shifted upward and downward from said center line.

In one embodiment, a rotation reduction ratio is applied on said hourglass surface interlocking with said mating components to increase applied forces.

In one embodiment, said second pair of actuation wires passed through a hole in center of said center bevel gear (106).

This invention provides a non-back driven cable driven gripper 100. In the embodiments of present application, the instrument disclosed is a suturing device for flexible robots in endoscopic surgery. The device comprises of a gripping section 114 and a rotation section 115.

In the embodiments of present application, the two pairs of actuation wire 112, 113 come from the instrument channel of the flexible robotic arm, in a pull-pull configuration, making downscale of the whole structure possible. The softness of the wire enables bending without exerting significant force to the structure.

The gripping section 114 comprises of globoid worm gear 103 with mirrored thread and two gripping jaws 101, 102 with gear teeth. Driving the globoid worm gear 103 control the angle of both gripping jaws 101, 102. These components are enclosed by a circular housing 108, 109.

The rotation section 115 comprises of two small driving bevel gears 104, 105 and one center bevel gear 106 connected to the housing of the gripping section 108, 109. Driving one of the small bevel gears 104, 105 turns the center bevel gear 106 as well as another small bevel gear 104, 105. These components are enclosed by another circular housing 110, 111.

The end-effecter on the robot instrument can be a gripper 101, 102 of any form, for example with serrated face or features that fits specific suturing needle.

This is the detailed description for the structure of the non-back driven cable driven gripper 100 and the mechanism for driving the gripper section 114 and the rotation section 115 respectively.

In the rotation section 115, the rotational motion is controlled by pair of actuation wire 112, at which one wire is connected to a small bevel gear 105, and one wire is connected to another small bevel gear 104. This pair of actuation wire 112 is actuated in a pull-pull mechanism such that the tension of the actuation wires is in the same direction, and the displacement of each actuation wire is the same but in a different direction. This mechanism enables the center bevel gear 106 connection perpendicularly with these two small bevel gears 104, 105 can be rotated along the center axis of the gripper from the pair of actuation wire 112. The supporting bearing 107 ensures the center bevel gear 106 can be rotated steadily inside the rotation section 115 and the housings 110, 111.

In the gripping section 114, another pair of actuation wire 113 is used to control the motion of the gripper jaws 101, 102. The pair of actuation wire 113 passes through the small hole on the axis of the center bevel gear 106 and it is connected at the two opposite of the globoid worm gear 103. With the same pull-pull mechanism acting on the actuation wire 113, the globoid worm gear 103 rotates along its own axis accordingly; supported by the gripper section housing 108, 109. The globoid worm gear 103 has two sets of tongues and groves along the hourglass surface, which are used to drive the duality gripper to manipulate flesh or tools for suturing. The specific geometric configuration is a globoid spiral, this configuration allows the center of rotation of both the driving and driven bodies to remain in the same position while maintain full gear surface contact. This is critical to be able to miniaturize component size as well as allow component to be micromachined. These tongue and grove surfaces interlock with a single gear on the mating components, gripper jaws 101 and 102. The two spirals rotate against each other driving the two bodies to simultaneously increase or decrease gripping angle. The gripping bodies only move upon receiving an input force to change gripper positions as tongue and grove spiral force the bodies to lock in position. The spiral also amplifies force by applying rotational reduction ratio. This increased force can be used to grip any suturing needle. Instead of placing in the middle, the gear teeth on gripper jaws 101 and 102 are shifted upwards and downwards and designed to fit the new grove profile. This configuration prevents interference, simplifies the geometry and improves the strength of the model. Various surface features can be integrated on the gripper surface to better secure suture instruments or flesh.

The rotation section 115 is held within bottom rotation section housing 110 and top rotation section housing 111. The gripping section 114 is held within bottom gripper section housing 108 and top gripper section housing 109. The rotation section housing 110 and 111 have four mounting holes for metal pins and the gripper section housing 108 and 109 have three mounting holes for metal pins. Centre bevel gear 106 is connected to bottom gripper section housing 108 and top gripper section housing 109 by pinning the two holes at its end. At the front of the bottom rotation section housing 110, a small tongue that can slide between grove on globoid housing 108 and 109 act as a stopper. The tongue is 90 degrees wide and the grove is 270 degrees wide, making a maximum range of 180 degree. On the other hand, the range of gripper is also limited by the gripper section housing 108 and 109. To facilitate smoother wiring and easier manufacturing, the two wiring holes on bottom gripper section housing 109 are pointing towards the of the center line of the whole assembly. Because the pair of actuation wire 113 is not directly attached to gripper jaws 101 and 102, they can be taken out simply by removing one metal pin. This is useful because different types of jaws can be used for the same robotic arm, thus saving cost and effort. In the assembly process, all moving components can be installed into bottom rotation section housing 110 and bottom gripper section housing 108 before closing with the top rotation section housing 111 and top gripper section housing 109. At the back of the rotation section housing 110 and 111, the four outermost holes are for anchoring four wires from the flexible robotic arm.

For the structure of the small bevel gear 104 and 105, a wiring grove under the gear face is presented. The small curvature of the grove is best utilized when the actuation wire is soft but tough. At the end of the grove, a straight hole is cut to reach a larger opening. A hollow metal piece with wire passing through is pressed tightly and the piece helps anchoring the wire in the opening.

1. An end-effector for a robotic arm, comprising:
a. a first housing having a center line;
b. at least one rotational member with rotational axis perpendicular to said center line;
c. a first pair of actuation wires;
wherein said first pair of actuation wires are connected to said at least one rotational member to actuate rotation with a pull-pull mechanism whereby tension and displacement in each wire of said first pair of actuation wires are same but direction is different.

2. The end-effector of claim 1, wherein said at least one rotational member comprises two bevel gears (104, 105), each of said two bevel gears is separately connected to one of said first pair of actuation wires (112) to form said pull-pull mechanism, said two bevel gears connects to a central bevel gear (106) with a rotational axis along said center line.

3. The end-effector of claim 1, wherein said at least one rotational member comprises a globoid worm gear (103) having two ends, wherein each of said two ends is separately connected to one of said first pair of actuation wires (113) to form said pull-pull mechanism.

4. The end-effector of claim 3, wherein said globoid worm gear (103) comprises an hourglass surface having two sets of tongues and groves interlocking with mating components on gripper jaws (101, 102).

5. The end-effector of claim 2, wherein said central bevel gear (106) is connected to an end part to actuate rotation of said end part.

6. The end-effector of claim 5, wherein said end part comprises a second housing and a second pair of actuation wires, wherein said second housing contains a globoid worm gear (103) having two ends, wherein each of said two ends is separately connected to one of said second pair of actuation wires (113) to form a pull-pull mechanism.

7. The end-effector of claim 6, wherein said globoid worm gear (103) comprises an hourglass surface interlocking with mating components on two gripper jaws (101, 102) to drive angle of said two gripper jaws.

8. The end-effector of claim 6, wherein said first housing comprises a tongue for insertion into a grove on said second housing to act as a stopper.

9. The end-effector of claim 2, wherein said two bevel gears comprises a gear face and a wiring grove, wherein said wiring grove is under said gear surface.

10. The end-effector of claim 4, wherein said gripper jaws are connected to said end-effector with one or more metal pins.

11. The end-effector of claim 4, wherein said mating components comprises a small gear.

12. The end-effector of claim 4, wherein said hourglass surface comprises two sets of tongues and groves, each of said two sets of tongues and groves being in opposite direction of the other.

13. The end-effector of claim 4, wherein said two gripper jaws comprises gear teeth shifted upward and downward from said center line.

14. The end-effector of claim 4, wherein a rotation reduction ratio is applied on said hourglass surface interlocking with said mating components to increase applied forces.

15. The end-effector of claim 2, wherein said second pair of actuation wires passed through a hole in center of said center bevel gear (106).

16. The end-effector of claim 7, wherein said gripper jaws are connected to said end-effector with one or more metal pins.

17. The end-effector of claim 7, wherein said mating components comprises a small gear.

18. The end-effector of claim 7, wherein said hourglass surface comprises two sets of tongues and groves, each of said two sets of tongues and groves being in opposite direction of the other.

19. The end-effector of claim 7, wherein said two gripper jaws comprises gear teeth shifted upward and downward from said center line.

20. The end-effector of claim 7, wherein a rotation reduction ratio is applied on said hourglass surface interlocking with said mating components to increase applied forces.
